(19) United States
(12) Patent Application Publication
(10) Pub. No.: US 2026/0199646 A1
(43) Pub. Date: Jul. 16, 2026

(54) SYSTEMS AND METHODS FOR SELECTIVELY ADJUSTING IMPLANTABLE SHUNTING SYSTEMS

(71) Applicant: Shifamed Holdings, LLC, Campbell, CA (US)

(72) Inventors: Brian Fahey, Menlo Park, CA (US); Anthony Pantages, San Jose, CA (US); Scott Robertson, Portland, OR (US); Miles Alexander, Fremont, CA (US)

(21) Appl. No.: 19/136,857

(22) PCT Filed: Dec. 20, 2023
(86) PCT No.: PCT/US2023/085145

§ 371 Date: Jun. 6, 2025

Related U.S. Application Data
(60) Provisional application No. 63/476,685, filed on Dec. 22, 2022.

(51) Int. Cl. A61M 27/00

(52) U.S. Cl. CPC A61M 27/002; A61M 2205/0266; A61M 2205/3334; A61M 2210/125

(57) ABSTRACT

The present technology generally relates to implantable medical devices and, in particular, to systems and methods for selectively adjusting implantable shunting systems, including fully closing the shunting systems after implantation within a patient. For example, many embodiments of the present technology are directed to systems and method for plugging or sealing a lumen in an adjustable shunting element configured to fluidly couple a first body region and a second body region. At least some embodiments of the systems and methods for selectively closing interatrial shunting systems include closure devices configured to be removable, such that the closure device can be removably coupled to the implantable shunting system to at least partially or fully prevent fluid flow therethrough for a time, and can be removed from the implantable shunting system to allow fluid flow through the implantable shunting system to resume.

### CROSS-REFERENCE TO RELATED APPLICATION(S)

This application claims priority to U.S. Provisional App. No. 63/476,685, filed Dec. 22, 2022, the disclosure of which is incorporated herein by reference in its entirety.

### TECHNICAL FIELD

The present technology generally relates to implantable medical devices and, in particular, to systems and methods for selectively adjusting implantable shunting systems, including fully closing the shunting systems after implantation.

### BACKGROUND

Shunting systems have been widely proposed for treating various disorders associated with fluid build-up or pressure in a particular body region. For example, interatrial shunting systems that shunt blood from the left atrium of the heart to the right atrium of the heart have been proposed as a treatment for heart failure in general, and heart failure with preserved ejection fraction in particular. Proposed shunting systems range in complexity from simple tube shunts to more sophisticated systems having on-board electronics, adjustable lumens, or the like. Despite the advancement of shunting system technology, designing shunting systems that can be reliably and relatively non-invasively delivered and deployed across a target structure remains a challenge.

### BRIEF DESCRIPTION OF THE DRAWINGS

FIG. 1 is an illustration of an interatrial device implanted in a heart and configured in accordance with select embodiments of the present technology.

FIG. 2 is a partially-schematic illustration of an adjustable interatrial shunting system configured in accordance with select embodiments of the present technology.

FIG. 3 is a cross-sectional illustration of an adjustable interatrial shunting system including one or more closing members configured in accordance with embodiments of the present technology.

FIG. 4 is a cross-sectional illustration of an adjustable interatrial shunting system and a plug configured in accordance with embodiments of the present technology.

FIG. 5A is a side view of a closing device configured in accordance with embodiments of the present technology.

FIG. 5B is a perspective end view of the closing device of FIG. 5A.

FIG. 6 is an illustration of an adjustable interatrial shunting system and a closing device configured in accordance with embodiments of the present technology.

FIGS. 7A and 7B are partially-schematic illustrations of an adjustable interatrial shunting system including one or more closing stops configured in accordance with embodiments of the present technology.

FIG. 8A is an illustration of an adjustable interatrial shunting system and a closing device configured in accordance with embodiments of the present technology.

FIG. 8B is an illustration of the closing device of FIG. 8A and a delivery tool configured in accordance with embodiments of the present technology.

FIG. 9 is a cross-sectional illustration of an adjustable interatrial shunting system including one or more closure retention features configured in accordance with embodiments of the present technology.

FIG. 10A is an illustration of an adjustable interatrial shunting system and a closing cap configured in accordance with embodiments of the present technology.

FIG. 10B is an illustration of the closing cap of FIG. 10A and a delivery tool configured in accordance with embodiments of the present technology.

FIGS. 11A and 11B are illustrations of an adjustable interatrial shunting system including closing portions and closing levers configured in accordance with embodiments of the present technology.

FIGS. 12A and 12B are illustrations of an adjustable interatrial shunting system including a cartridge configured to contain a sealing compound configured in accordance with embodiments of the present technology.

FIGS. 13A and 13B are illustrations of an interatrial shunting system configured in accordance with embodiments of the present technology.

FIGS. 14A and 14B are partially-schematic illustrations of an adjustable interatrial shunting system including a restricting portion configured in accordance with embodiments of the present technology.

### DETAILED DESCRIPTION

The present technology is directed to implantable medical devices, systems, and methods for selectively adjusting (e.g., restricting, closing, or blocking) fluid flow through implantable shunting systems. For example, many embodiments of the present technology are directed to systems and method for plugging or sealing a lumen in a shunting element configured to fluidly couple a first body region and a second body region. Additionally, or alternatively, at least some of the systems and method for closing implantable shunting systems described herein can be configured to be coupled to fixed or otherwise non-adjustable shunts. In these and other embodiments, the systems and methods for selectively closing interatrial shunting systems include closure devices configured to be removable, such that the closure device can be removably coupled to the implantable shunting system to at least partially or fully prevent fluid flow therethrough for a time, and can be removed from the implantable shunting system to allow fluid flow through the implantable shunting system to resume. Various aspects of the implantable shunting system and/or the associated therapy can be adjusted when the closure device is coupled to the implantable shunting system.

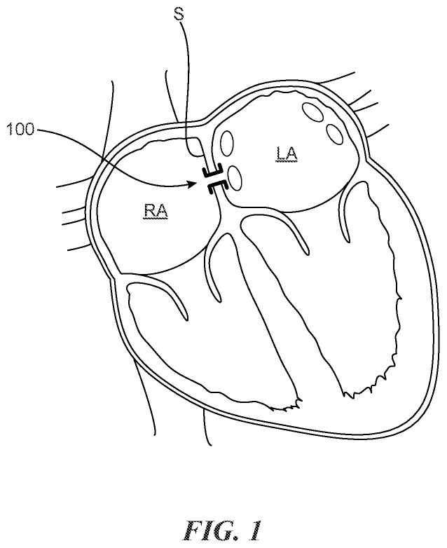

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-14B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately.” and “about” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a left atrium of a heart) and a second region (e.g., a right atrium or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system. For example, any of the shunts described herein, including those referred to as “interatrial,” may be nevertheless used and/or modified to shunt blood between the left atrium (“LA”) and the coronary sinus, or between the right pulmonary vein and the superior vena cava. Moreover, while the disclosure herein primarily describes shunting blood from the LA to the right atrium (“RA”), the present technology can be readily adapted to shunt blood from the RA to the LA to treat certain conditions, such as pulmonary hypertension. For example, mirror images of embodiments, or in some cases identical embodiments, used to shunt blood from the LA to the RA can be used to shunt blood from the RA to the LA in certain patients. Moreover, while certain embodiments herein are described in the context of heart failure treatment, any of the embodiments herein, including those referred to as interatrial shunts, may nevertheless be used and/or modified to treat other diseases or conditions, including other diseases or conditions of other body regions. For example, the systems described herein can be used to treat diseases characterized by increased pressure and/or fluid build-up, including but not limited to glaucoma, pulmonary failure, renal failure, hydrocephalus, and the like.

Although certain aspects of the present technology are described with reference to closing or otherwise adjusting fluid flow and/or a flow resistance at a left atrial portion or a right atrial portion of an interatrial shunt device, a person of ordinary skill in the art will appreciate that the present technology can be used to close or otherwise adjust flow/flow resistance at other portions of the interatrial shunt device. For example, at least some embodiments of the present technology can be used to close and/or otherwise adjust flow/flow resistance in the right atrial portion of the interatrial shunt device, the left atrial portion of the interatrial shunt device, and/or another portion of the interatrial shunt device.

Although certain aspects of the present technology are described with reference to closing or otherwise adjusting fluid flow and/or a flow resistance through an implantable device-based interatrial shunt, a person of ordinary skill in the art will appreciate that the present technology can be used to close or otherwise adjust flow/flow resistance through other shunts and/or shunting systems. For example, at least some embodiments of the present technology can be used to close and/or otherwise adjust flow/flow resistance through procedure-based interatrial shunts, and/or other suitable interatrial shunts.

The headings provided herein are for convenience only and do not interpret the scope or meaning of the claimed present technology.

### A. Interatrial Shunts for Treatment of Heart Failure

Interatrial shunts have recently been proposed as a way to reduce elevated left atrial pressure, and this emerging class of cardiovascular therapeutic interventions has been demonstrated to have significant clinical promise in treating patients with heart failure. FIG. 1 shows the placement of a shunt in the septal wall between the LA and RA. Most interatrial shunts (e.g., shunt 100) involve creating a hole or inserting a structure with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

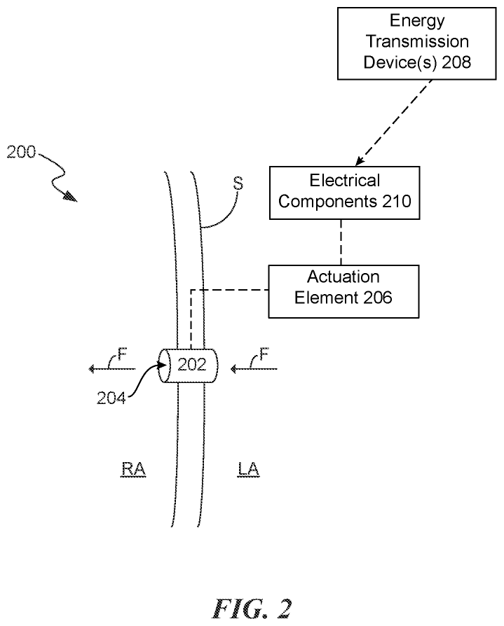

FIG. 2 is a schematic illustration of an interatrial shunting system 200 (“system 200”) configured in accordance with an embodiment of the present technology. The system 200 includes a shunting element 202 defining a lumen 204 therethrough. When implanted across the septal wall S, the system 200 can fluidly connect the LA and the RA via the lumen 204. Accordingly, when the shunting element 202 is implanted in the septal wall, blood can flow from the LA to the RA via the lumen 204 (as shown by arrows F). As illustrated in FIG. 2, the shunting elements 202 can extend beyond/protrude outwardly from the septal wall S into the LA and/or the RA, as opposed to congenital and/or iatrogenic atrial septal defects (“ASDs”) and/or patent foramen ovales (“PFOs”) which generally extend through but not beyond the septal wall S. The shunting element 202 can include additional features not shown in FIG. 2, such as a frame, anchors, membrane, or the like. For example, the shunting element 202 can include features such as those described in International Patent Application No. PCT/US2020/049996, the disclosure of which is incorporated by reference in its entirety.

The system 200 can further include an actuation element 206 configured to selectively change a geometry (size, shape, etc.) and/or other characteristic of the shunting element 202 to selectively modulate (e.g., increase or decrease) the flow of fluid through the lumen 204. For example, the actuation element 206 can be configured to adjust the shape and/or geometry of the lumen so that fluid flow through the lumen 204 is at least partially or fully prevented, for example, by selectively decreasing a diameter of the lumen 204 and/or otherwise reducing an internal cross-sectional area defined by the lumen 204.

In some embodiments, at least a portion of the actuation element 206 comprises a shape memory material, such as a shape memory metal or alloy (e.g., nitinol, including nitinol-based alloys), a shape memory polymer, or a pH-based shape memory material. In embodiments in which the actuation element is composed of a shape memory material (which may be referred to herein as a “shape memory actuation element”), the shape memory actuation element can be configured to change in geometry (e.g., transform between a first configuration and a second configuration) in response to a stimulus (e.g., heat or mechanical loading). The movement of the actuation element 206 can adjust the geometry of the lumen 204, as described above. Additional aspects of adjusting an interatrial shunt using shape memory actuation elements, including various adjustable interatrial shunts incorporating shape memory actuation elements, are described in International Application No. PCT/US2020/049996, previously incorporated by reference herein.

The system 200 can further include energy transmission device(s) 208 for delivering energy (e.g., power) to the implanted components (e.g., the actuation element 206 and/or the implanted electrical components 210, described below) of the system 200. The energy transmission device(s) 208 can include any device or system that is capable of transmitting energy to an implanted component, through a wired and/or wireless connection. For example, an energy transmission device 208 can be configured to transmit radiofrequency (RF) energy, microwave frequency energy, other forms of electromagnetic energy, ultrasonic energy, thermal energy, or other types of energy in accordance with techniques known to those of skill in the art.

The system 200 can further include electrical components 210 implanted with the shunting element 202 and electrically coupled together to form electrical circuits (e.g., RLC circuits, resonant circuits, etc.). The electrical components 210 can include, for example, conventional electrical components found in electrical circuits, such as resistors, capacitors, and inductors. The electrical components 210 can receive energy and/or power from the energy transmission device(s) 208. For example, in some embodiments, the energy transmission device(s) 208 generate an electromagnetic field, and the electrical components 210 generate an electrical current in response to being exposed to the electromagnetic field. The current generated by the electrical components 210 can flow through and directly provide power to (e.g., resistively heat) the actuation element 206. For example, the actuation element 206 can be incorporated into an electrical circuit formed by the electrical components 210 such that the energy transmission device(s) 208 can directly power the actuation element 206 by generating a current in the electrical circuit that flows through and resistively heats the actuation element 206.

### B. Selected Embodiments of Systems and Methods for Closing Implantable Shunting Systems

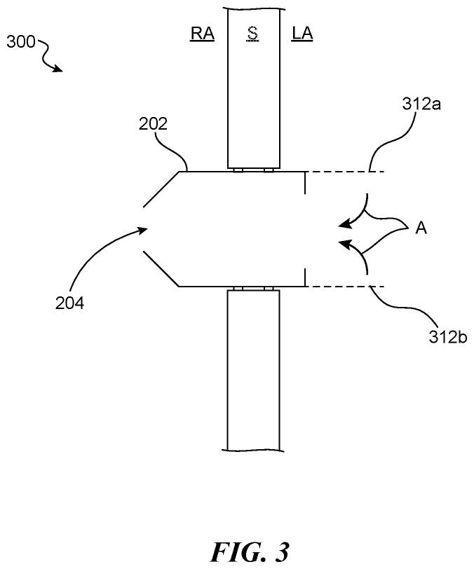

Systems and methods for at least partially or fully preventing flow through implantable shunting systems, including adjustable interatrial shunts, are described with reference to FIGS. 3-14B. At least some features of one or more of the embodiments described herein can be used together and/or combined with features from one or more of the other embodiments described herein. For example, the system 200 of FIG. 2 can include one or more features from one, two, or more of the embodiments described below with reference to FIGS. 3-14B. Additionally, or alternatively, individual ones of the embodiments described herein with reference to FIGS. 3-14B can be configured to gradually or titratably adjust fluid flow and/or resistance to fluid flow through the shunting element 202, e.g., in vivo or otherwise after the shunting element 202 has been implanted within a patient. In some embodiments, the shunting element 202 can be partially closed using one or more of the features described herein and, at some later time, reopened and/or used as a pathway to recross the septal wall (e.g., during a same or different procedure). In some embodiments, for example, the shunting element 202 can be at least partially or fully opened (e.g., by actuating the shunting element 202 to open, using a balloon or other expandable device to mechanically force the shunting element 202 to open, etc.) before being closed using one or more of the embodiments described herein. The systems and methods described herein may also be used to close (e.g., fully close, partially close, stenose, etc.) or otherwise adjust fluid flow through adjustable shunting systems and/or fixed or otherwise non-adjustable shunting systems, including fixed or non-adjustable interatrial shunts. As described above with reference to FIG. 2, at least some aspects of the system 200 can be structurally and/or geometrically different than other openings or holes through the septal wall S, including the openings/holes associated with ASDs and/or PFOs. For at least these reasons, devices and/or methods for closing ASDs and/or PFOs are expected to have little to no applicability to the problem of closing implantable shunting systems, including the implantable shunting systems of the present technology.

FIG. 3 is a cross-sectional illustration of an interatrial shunting system 300 (“system 300”) configured in accordance with embodiments of the present technology. The system 300 can include at least some features that are at least generally similar or identical in structure and/or function to the system 200 of FIG. 2. In at least some embodiments, for example, the system 300 includes the shunting element 202 and lumen 204 therethrough. Additionally, the system 300 can include one or more closing members 312 (individually identified as a first closing member 312a and a second closing members 312b in FIG. 3). Although the illustrated system 300 includes two closing members 312, in other embodiments the system 300 can include more or fewer closing members 312, such as at least 1, 3, 4, 5, 6, or another suitable number of closing members 312. Each of the closing members 312 can have a square, rectangular, circular, arcuate, or other suitable shape. Each of the closing members 312 can be configured to transition between (i) a first orientation or state in which the closing member(s) 312 allows fluid flow through the lumen 204 and (ii) a second orientation or state in which the closing member(s) 312, reduces, prevents, or substantially prevents fluid flow through the lumen 204. In at least some embodiments, one or more of the closing members 312 can be formed from a shape memory metal or alloy, including any of the shape memory materials described herein. In such embodiments, the system 200 can be implanted in with the closing members 312 in the first configuration/state (e.g., at a given lumen 204 or lumen orifice size), and the closing members 312 can be actuated (e.g., via internally and/or externally applied energy) to transition the closing members 312 toward and/or to the second configuration/state to reduce or prevent flow through the lumen 204. In the illustrated embodiment, the closing members 312 are configured to pivot or rotate toward the lumen 204, in the direction shown by arrow A, to transition from the first state toward the second state. In other embodiments, the closing members 312 define an iris and are configured to pivot or rotate radially inwardly toward a longitudinal axis of the shunting element 202, for example, irising from the first state toward and/or to the second state to prevent or substantially prevent fluid flow through the lumen 204. In these and other embodiments, each of the closing members 312 can be configured to pivot or rotate up to or at least 15 degrees, 30 degrees, 45 degrees, 60 degrees, 90 degrees, 105 degrees, 120 degrees, 135 degrees, 150 degrees, 165 degrees 180 degrees, any angle therebetween, or another suitable angle when transitioning between the first state and the second state. Although the closing members 312 are positioned on a left atrial LA side of the shunting element 202, in these and other embodiments the shunting element 202 can include one or more closing members 312 on a right atrial RA side of the shunting element 202. In some embodiments, one or more of the closing member(s) 312 can be covered, or at least partially covered, by one or more biocompatible materials, which can increase an effective surface area of the one or more closing member(s) 312 and/or improve the flow-preventing performance of the closing member(s) 312 in the second state.

In some embodiments, the closing members 312 can be configured to partially close the opening, e.g., to a diameter of between about 0.5 mm and about 4 mm, such as 2 mm or another suitable diameter. At this size, the shunting element 202 is expected to become stenotic via tissue overgrowth in a short period of time (e.g., within hours, days, or weeks). This, in turn, can allow the shunting element 202 to serve as a “target” site or pathway that would allow a user to recross the septal wall via the shunting element 202, e.g., at some later time. For example, the user could traverse the shunting element 202 and, using a guidewire and balloon, could at least partially or fully re-open the shunting element 202, if desired. In at least some embodiments, for example, the user can reopen or otherwise enlarge the shunting element 202 and use it as a temporary left heart access port, and may reclose or return the shunt to its small/closing size upon completion of left heart access.

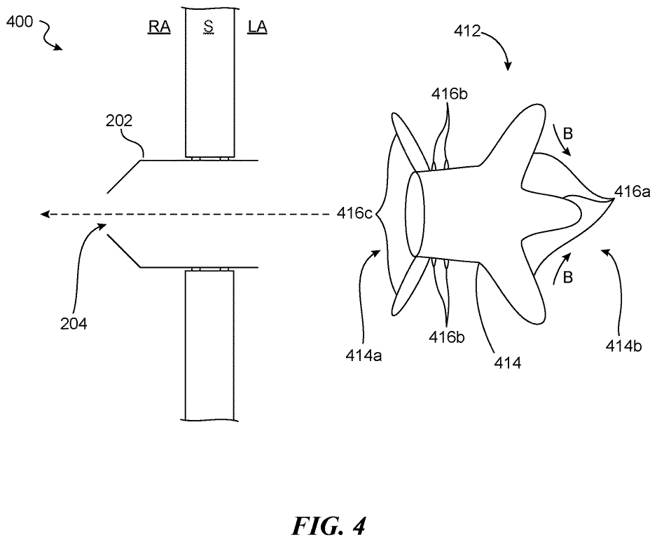

FIG. 4 is a cross-sectional illustration of an interatrial shunting system 400 (“system 400”) configured in accordance with embodiments of the present technology. The system 400 can include at least some features that are at least generally similar or identical in structure and/or function to the system 200 of FIG. 2. In at least some embodiments, for example the system 400 includes the shunting element 202 defining the lumen 204 therethrough. In some embodiments, the shunting element 202 of the system 400 can be configured to receive a plug or closing member 412 (“plug 412”), e.g., while the shunting element 202 is in vivo or otherwise after the shunting element 202 has been implanted. The plug 412 can include a body 414 having a first or right atrial end portion 414a and a second or left atrial end portion 414b opposite the first end portion 414a. When received within the lumen 204, the first end portion 414a can be positioned on a first side of the septal wall S (e.g., within the RA) and the second end portion 414b can be positioned on a second side of the septal wall S opposite the first side (e.g., within the LA). In some embodiments, the body 414 can be configured to expand, for example, to at least partially or fully prevent movement of the plug relative to the shunting element 202 and/or fluid flow through the lumen 204 when all or a portion of the plug 412 is positioned within the lumen 204. Additionally, or alternatively, the body 414 can include one or more retention components 416 (individually identified as one or more first retention components 416a, one or more second retention components 416b, and one or more third retention components 416c in FIG. 4). Individual ones of the retention components 416 can coupled to the first end portion 414a, the second end portion 414b, and/or another portion of the body 414, and can be configured to contact or otherwise engage (e.g., operably engage) at least a portion of the shunting element 202 to at least partially or fully prevent movement of the plug 412 relative to the shunting element 202. In the illustrated embodiment, the first retention components 416a are arms or fingers configured to contact a left atrial LA portion of the shunting element 202 and/or the LA side of the septal wall to at least partially or fully prevent movement of the plug 412 in toward the right atrium RA. The first retention components 416a can extend radially outward from the plug 412, and can be deformable or otherwise configured to bend or deflect radially inwardly, such as in the direction indicated by arrows B in FIG. 4. Additionally, or alternatively, the shunting element 202 can include the second retention components 416b, individual ones of which can include spikes or other features configured to engage an inner portion of the shunting element 202, e.g., an inner portion or surface that at least partially defines the lumen 204. In these and other embodiments, the shunting element 202 can include the third retention components 416c, individual ones of which can include arms, loops, springs, or other features configured to engage a right atrial RA side of the of the shunting element 202. In the illustrated embodiment, the first end portion 414a includes the third retention components 416c, the second end portion 414b includes the first retention components 416a, and the second retention components 416b are positioned between the first retention components 416a and the third retention components 416c. In other embodiments, however, individual ones of the retention components 416 can have other positions along the body 414. In embodiments, one or more of the retention components 416 can be movable between relatively collapsed configurations (e.g., to facilitate percutaneous delivery in a desirable profile size) and relatively expanded configurations (e.g., to sufficiently act as a stabilization or retention feature).

In some embodiments, the energy transmission devices 208 (FIG. 2) can be used to actuate the shunting element 202 open before the plug 412 is positioned within the lumen. Opening the shunting element 202 is expected to make it easier to close the shunting element 202, for example, by placing the shunt in a configuration with a more uniform or consistent (e.g., cylindrical, or at least generally cylindrical) geometry. It is expected to be easier to position closing objects within the lumen 204 when the lumen 204 has this uniform/cylindrical shape.

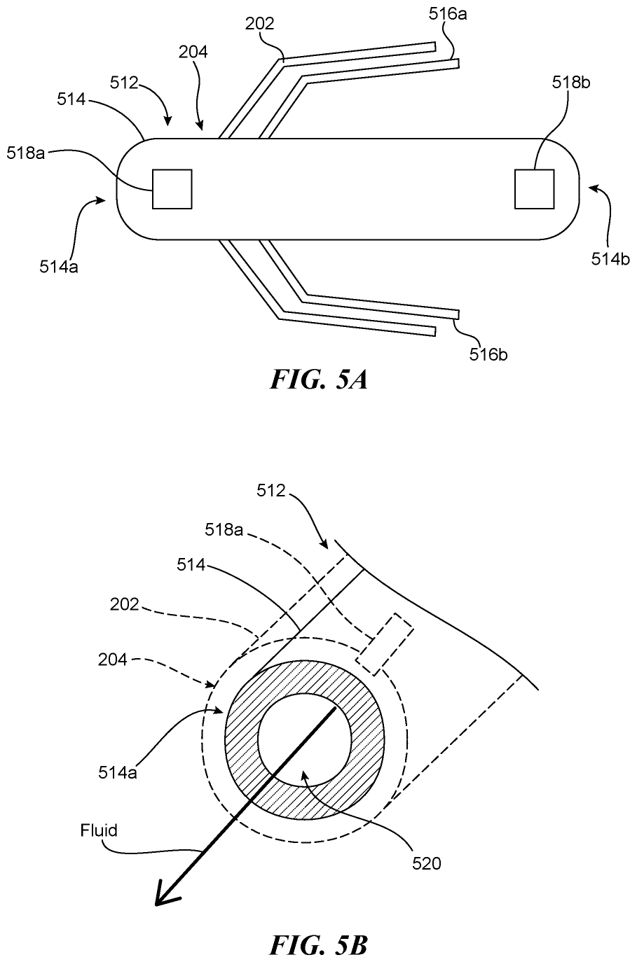

FIG. 5A is a side view of a closing device 512 (“device 512”) configured in accordance with embodiments of the present technology. The device 512 includes a body 514 having a first or right atrial end portion 514a and a second or left atrial end portion 514b opposite the first end portion 514a. The body 514 can be configured to be positionable at least partially within the lumen 204 of the shunting element 202, to at least partially or fully prevent fluid flow therethrough. In some embodiments, the device 512 can include one or more retention elements 516 (individually identified as a first retention element 516a and a second retention element 516b in FIG. 5A) configured to contact or otherwise engage the shunting element 202 to at least partially or fully prevent movement of the device 512 relative thereto. In the illustrated embodiment, the retention elements 516 are spring arms biased outwardly from the body 514 and configured to press against an interior portion of the shunting element 202, but are shown as being spaced apart from the interior portion of the shunting element 202 for illustrative clarity. In other embodiments, the retention elements 516 can have another suitable configuration. In these and other embodiments, the device 512 can include one or more sensors or sensing components 518 (individually identified as a first sensor 518a and second sensor 518b in FIG. 5A). Each of the sensors 518 can include one or more temperature sensors, flow sensors, conductivity sensors, pressure sensors, and/or other suitable sensors. In the illustrated embodiment, the first sensor 518a is positioned at or proximate the first end portion 514a and the second sensor 518b is positioned at or proximate the second end portion 514b such that, when the device 512 is positioned within the lumen 204 of shunting element 202, the first sensor 518a can face toward and/or be positioned at least partially within the patient's right atrium and/or the second sensor 518b can face toward and/or be positioned at least partially within the patient's left atrium.

FIG. 5B is a perspective view of the right atrial end 514a of the device 512 of FIG. 5A positioned within the lumen 204 of the shunting element 202 (shown in dashed line), in accordance with embodiments of the present technology. The device 512 can at least partially or fully fill the lumen 204, e.g., to at least partially or fully prevent fluid flow therethrough. However, in some embodiments, the body 514 of the device 512 defines a lumen 520 through which fluid (e.g., blood) can flow. Accordingly, when the device 512 is positioned within the lumen 204, the lumen 520 can allow fluid to flow through the device 512 and the shunting element 202 but provide increased resistance to flow compared to the shunting element's lumen 204. Additionally, or alternatively, the sensors 518 (only the first sensor 518a is shown in FIG. 5B) can be configured to detect information associated with fluid within the lumen 520.

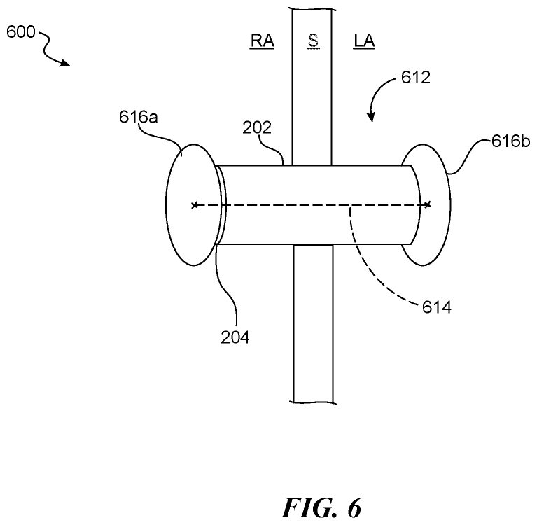

FIG. 6 is an illustration of an interatrial shunting system 600 (“system 600”) configured in accordance with embodiments of the present technology. The system 600 can include at least some features that are at least generally similar or identical in structure and/or function to the system 200 of FIG. 2. In at least some embodiments, for example, the system 600 includes the shunting element 202 and corresponding lumen 204 therethrough. Additionally, the system 600 can further include a closing device 612 configured to at least partially or fully prevent fluid flow through the shunting element 202. The closing device 612 can include one or more retention or sealing elements 616 (individually identified as a first retention element 616a and a second retention element 616b in FIG. 6) configured to contact or otherwise engage the shunting element 202, e.g., to at least partially or fully prevent fluid flow therethrough. One or more of the retention elements 616 can be expandable, deformable, and/or otherwise configured to conformably engage with the shunting element 202. In at least some embodiments, for example, the retention element 616 can be formed as a wireform, e.g., in a stent-like construction. In such embodiments, the retention element 616 can comprise filaments of extruded, rolled, and/or laser-cut construction. In these and other embodiments, one or more of the retention elements 616 can be configured to be transitioned between a first or compact delivery state/configuration and a second or expanded state/configuration. The transition between the first and second states may be facilitated by foreshortening of one or more portions of the retention element 616. In the second state, one or more of the retention elements 616 can complementarily engage the shunting element 202 to at least partially or fully prevent fluid flow through the lumen 204. In the illustrated embodiment, the first retention element 616a is configured to sealingly engage (e.g., form at least a substantially fluid-impermeable seal with) an RA portion of the shunting element 202 and the second retention element 616b is configured to sealingly engage a LA portion of the shunting element 202.

Additionally, or alternatively, one or more of the retention elements 616 can be expandable, e.g., to improve insertion through the lumen 204 and/or the sealing engagement with the shunting element 202. In at least some embodiments, for example, one or more of the retention elements 616 can include one or more expandable shape memory discs, balloons, foams, shape memory materials or alloys, or other suitable expandable structures such as non-shape memory wireforms and/or mesh structures that can be geometrically manipulated into an expanded state. In these and other embodiments, the retention elements 616 can be coupled to one another by a body including, e.g., one or more tethers or coupling members 614 (shown using dashed line in FIG. 6). The length of one or more of the tethers 614 can be approximately equal to or greater than a length of the shunting element 202, e.g., to allow the retention elements 616a-b to be positioned on opposite ends of the shunting element 202. In some embodiments, one or more of the tethers 614 can be elastic or otherwise configured to draw or bias the retention elements 616 toward one another. Accordingly, in the illustrated embodiment, one or more of the tethers 614 can be under tension when the first retention element 616a and the second retention element 616b are positioned on opposite sides of the shunting element 202, such that one or more of the tethers 614 can draw one or both of the first retention element 616a and the second retention element 616b toward one another and/or into sealing engagement with the respective end portions of the shunting element 202. In some embodiments, one or more of the tethers 614 can be initially placed in a state of relatively less tension (e.g., during a percutaneous delivery of device 612), and then manipulated into a state of relatively greater tension at a later timepoint. The manipulation of the tethers 614 can be done automatically (e.g., via decay of a biodegradable material, via a shape memory effect, etc.), via a user (e.g., by releasing a suture, tie, hook, and/or other binding element coupled to a delivery apparatus), and/or via other suitable arrangements and/or techniques. In some embodiments, the change in geometry of the one or more retention elements 616 is caused at least in part by a change in tension of the one or more tethers 614.

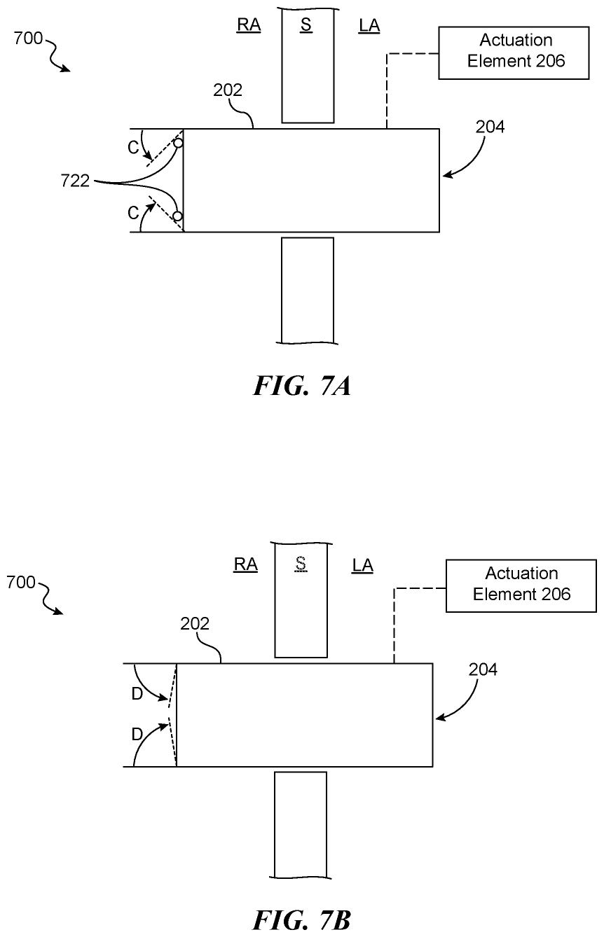

FIGS. 7A and 7B are partially-schematic illustrations of an interatrial shunting system 700 (“system 700”) configured in accordance with embodiments of the present technology. The system 700 can include at least some features that are at least generally similar or identical in structure and/or function to the system 200 of FIG. 2. In at least some embodiments, for example, the system 700 includes the shunting element 202 defining the lumen 204 therethrough. Referring first to FIG. 7A, in some embodiments, the system 700 can further include one or more closing stops or support components 722 (“stops 722”) configured to limit the adjustability of the shunting element 202 to a first range of adjustment, as shown by arrows C in FIG. 7A. In the illustrated embodiment, for example, the stops 722 are configured to physically and/or mechanically limit the adjustability of the shunting element 202. The first range of adjustment can change a fluid resistance of the shunting element 202 such that, independent of a state of the actuation element 206 and/or the shunting element 202, fluid is expected to flow through the lumen 204. The stops 722 can be removed, as shown in FIG. 7B, to increase the adjustability of the shunting element 202 to a second range of adjustment, as shown by arrows D in FIG. 7B, greater than the first range of adjustment (FIG. 7A). The second range of adjustment can allow the actuation element 206 to transition the shunting element 202 to a state in which fluid flow through the lumen 204 is substantially or fully prevented.

In some embodiments one or more of the stops 722 can include one or more tubular sections or annular components that extend circumferentially (either continuously or intermittently) about the lumen 204, e.g., at or near an opening to the lumen 240. Additionally, or alternatively, one or more of the stops 722 can include a shape memory material, can be initially placed in a wider (expanded) configuration (e.g. as in FIG. 7A), and can be narrowed (allowing the actuation element 206 to reach a more narrow geometry) by an operator by applying a compressive force (e.g., via a non-compliant balloon). Heating the stops above a phase transition temperature (e.g., via a percutaneously delivered energy source) can return individual ones of the stops 722 to the expanded configuration. In other embodiments, one or more of the stops 722 can be configured to operate in reverse, e.g., such that heating the stops 722 above a phase transition temperature narrows the stops 722 from the expanded configuration toward and/or to the narrowed configuration. In these and other embodiments, one or more of the stops 722 can include a membrane (e.g., a urethane, polymer, etc.) that encases another material (e.g., a bioabsorbable material, a fluid, etc.). Disrupting the membrane can cause the encased material to escape or otherwise degrade (e.g., immediately or over a predetermined amount of time), thereby allowing individual ones of the stops 722 to narrow and, in turn, increasing the degree to which the actuation element 206 can narrow/close the shunt opening geometry.

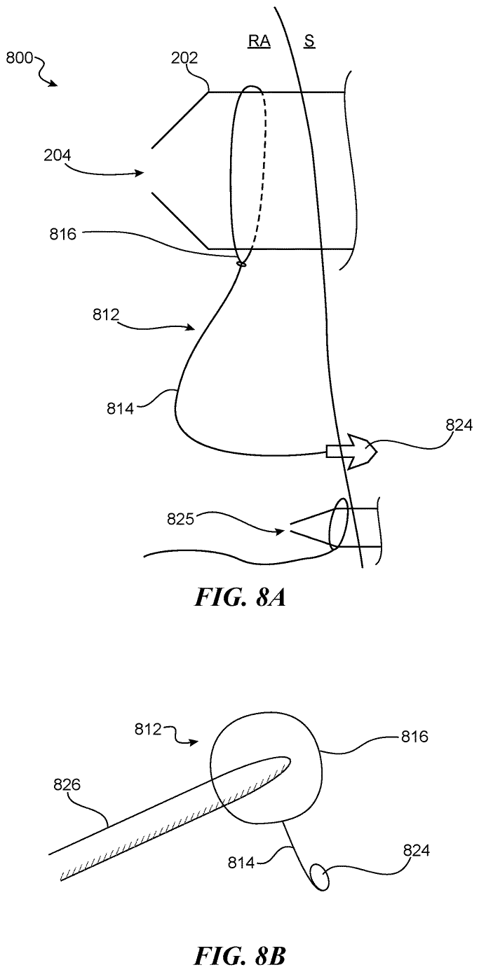

FIG. 8A is an illustration of an interatrial shunting system 800 (“system 800”) configured in accordance with embodiments of the present technology. The system 800 can include at least some features that are at least generally similar or identical in structure and/or function to the system 200 of FIG. 2. In at least some embodiments, for example, the system 800 includes the shunting element 202 and corresponding lumen 204. Additionally, the system 800 includes a closing device 812 configured to at least partially or fully prevent fluid flow through the shunting element 202. The closing device 812 includes a retention or sealing component 816 configured to extend around all or a portion of a perimeter/circumference of the shunting element 202. Although the sealing component 816 extends around a right atrial RA portion of the shunting element 202 in the embodiment illustrated in FIG. 8A, in other embodiments the sealing component 816 can extend around all or a left atrial or other suitable portion of the shunting element 202. The sealing component 816 can include a loop, a lasso, and/or other component configured to cinch or tighten around the shunting element 202 to reduce a diameter/shape of the lumen 204 and/or otherwise at least partially or fully prevent fluid flow therethrough. In some embodiments, a biocompatible sleeve or membrane can be positioned around at least a portion of the shunting element 202, at least partially between the sealing component 816 and the shunting element 202, such that the sealing component 816 can cinch or tighten around the biocompatible sleeve or membrane, and the biocompatible sleeve or membrane can in turn reduce or prevent flow through the shunting element 202. The sealing component 816 can be configured to tighten automatically (e.g., in response to heat or received from the energy transmission device(s) 208 in FIG. 2) or manually (e.g., during a procedure performed by a practitioner). In some embodiments, the sealing component 816 can be anchored to patient anatomy, such as the septal wall S or other suitable patient tissue. In the illustrated embodiment, for example, the closing device 812 includes an anchor 824 at least partially or fully embedded within or otherwise coupled to the septal wall S, and the sealing component 816 is coupled to the anchor by one or more tethers 814. The anchor 824 can be configured to help maintain tension on the tethers 814 and/or the sealing component 816, e.g., to keep the sealing component 816 cinched or tightened around the shunting element 202. Optionally, one or more anchor receiving components 825 can be at least partially or fully embedded within or otherwise coupled to the septal wall S, and the anchor 824 can be coupled to one or more of the anchor receiving components 825. In these and other embodiments, the closing device 812 can be positioned around the shunting element 202 using a delivery tool, such as the delivery tool 826 shown in FIG. 8B. Additionally, or alternatively, the delivery tool 826 can be used to tighten or untighten the sealing component 816, e.g., to increase or decrease a resistance to fluid flow through the lumen 204 (FIG. 8A).

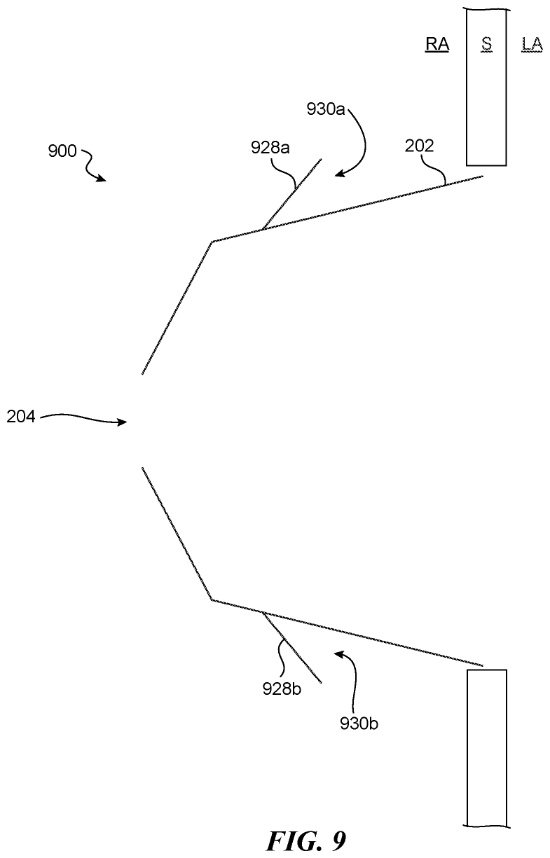

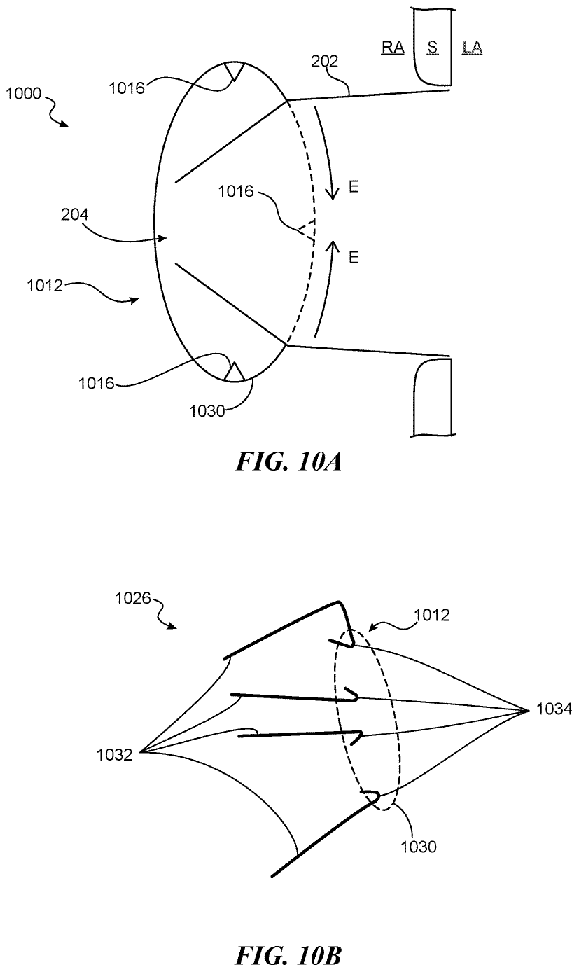

FIG. 9 is a cross-sectional illustration of an interatrial shunting system 900 (“system 900”) configured in accordance with embodiments of the present technology. The system 900 can include at least some features that are at least generally similar or identical in structure and/or function to the system 200 of FIG. 2. In at least some embodiments, for example, the system 900 includes the shunting element 202 and lumen 204. In some embodiments, the shunting element 202 can include one or more closure retention features 928 (individually identified as a first closure retention feature 928a and a second closure retention feature 928b in FIG. 9), each of which can extend radially outwardly (e.g., from the lumen 204) and/or be configured to releasably couple to or otherwise hold one or more closing or adjustment members or devices (not shown), such that the closing member(s) or device(s) can at least partially or fully prevent fluid flow through the lumen 204. In the illustrated embodiment, for example, each of the closure retention features 928a-b at least partially defines a capture zone or region 930 (individually identified as a first capture region 930a at least partially defined by the first closure retention feature 928a and a second capture region 930b at least partially defined by the second closure retention feature 928b in FIG. 9). At least a portion of the closing member(s) or device(s) used to prevent flow through the lumen 204 can be positioned within one or more of the capture regions 930 to couple/hold the closing member(s) or device(s) relative to the shunting element 202 and/or otherwise at least partially or fully prevent movement of the closing member(s) or device(s) relative to the shunting element 202. In at least some embodiments, for example, at least a portion of the sealing component 816 of FIGS. 8A and 8B can be positioned within one or more of the capture regions 930 to at least partially or fully prevent movement of the sealing component 816 relative to the shunting element 202, such as sliding movement of the sealing component 816 toward and/or off of a right atrial RA end of the shunting element 202. Another example of a closing device that can be coupled to/held by the closure retention features 928 is described in detail below with reference to FIGS. 10A and 10B.

FIG. 10A is an illustration of an interatrial shunting system 1000 (“system 1000”) configured in accordance with embodiments of the present technology. The system 1000 can include at least some features that are at least generally similar or identical in structure and/or function to the system 200 of FIG. 2. In at least some embodiments, for example, the system 1000 includes the shunting element 202 defining the lumen 204 therethrough. Additionally, the system 1000 can further include a closing device or cap 1012. The cap 1012 is configured to be positioned around and/or coupled to at least a portion of the shunting element 202, such as a right atrial RA end of the shunting element 202, to at least partially or fully prevent fluid flow through the lumen 204. The cap 1012 may comprise one or more meshes, foams, and/or other suitable materials.

In some embodiments, the cap 1012 can include one or more retention features 1016 configured to releasably couple the cap 1012 to the shunting element 202. In the illustrated embodiment, the retention features 1016 are spikes or teeth positioned around a perimeter or opening 1030 of the cap 1012. Additionally, or alternatively, the cap 1012 can include other retention features 1016 and/or retention features 1016 at other suitable positions. In these and other embodiments, individual ones of the retention features 1016 and/or one or more other portions of the cap 1012 can be configured to at least partially prevent, fully prevent, and/or otherwise reduce or prevent the impact of tissue overgrowth on the operation of the system 1000, such as tissue overgrowth that may alter or prevent the coupling of the cap 1012 to the shunting element 202. In at least some embodiments, for example, coupling the cap 1012 to the shunting element 202 can disrupt tissue growth, such as by removing or penetrating all or a portion of the overgrown tissue. In the illustrated embodiment, the cap 1012 at least partially or fully prevents fluid flow through the lumen 204 by covering or blocking all or a portion of the right atrial RA end of the shunting element 202. Additionally, or alternatively, the cap 1012 can be configured to apply a compressive and/or circumferential force, as indicated by arrows E, to at least a portion of the shunting element 202, such as a right atrial RA end portion of the shunting element 202, to at least partially or fully prevent fluid flow through the lumen 204.

FIG. 10B is an illustration of the cap 1012 and a delivery tool 1026 configured in accordance with embodiments of the present technology. The delivery tool 1026 can include one or more arms 1032 (four shown in FIG. 10B), and each of the arms 1032 can include a carrying or delivery member 1034 configured to hold the cap 1012. In the illustrated embodiment, for example, each of the carrying members 1034 include a hook configured to be positioned at least partially around the perimeter of the cap 1012. Additionally, or alternatively, one or more of the carrying members 1034 can have other suitable configurations. In these and other embodiments, the delivery tool 1026 can be used to position the cap 1012 relative and/or around all or a portion of a shunting element, such as the shunting element 202 of FIG. 10A. Individual ones of the carrying members 1034 can be decoupled or disconnected from the perimeter 1030 when the cap 1012 is positioned around the shunting element, e.g., to release the cap 1012 from the delivery tool 1026. In some embodiments, one or more of the carrying members 1034 can be adapted to interface with a feature of the shunting element 202 (FIG. 10A) with one or more of the retention features 1016, and/or another portion of the cap 1012. In these and other embodiments, one or more of the carrying members 1034 and/or one or more of the retention features 1016 can be configured to interface with a feature on the shunting element 202 that is at least generally similar or identical in structure and/or function to one or more of the features described elsewhere herein (e.g., the retention feature 928 of FIG. 9, the capture region 930 of FIG. 9, etc.).

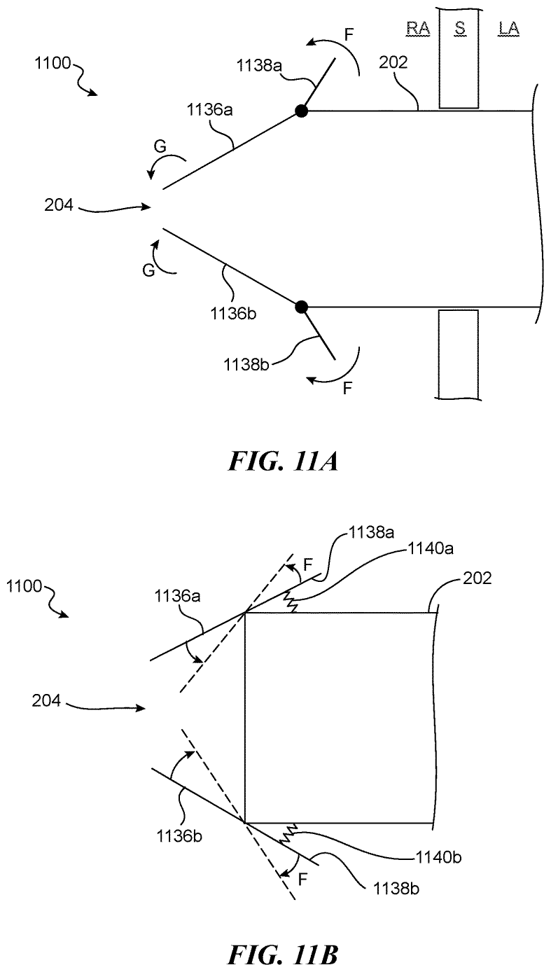

FIG. 11A is an illustration of an interatrial shunting system 1100 (“system 1100”) configured in accordance with embodiments of the present technology. The system 1100 can include at least some features that are at least generally similar or identical in structure and/or function to the system 200 of FIG. 2. In at least some embodiments, for example, the system 1100 includes the shunting element 202 and corresponding lumen 204. In some embodiments, the shunting element 202 includes one or more closing portions or members 1136 (individually identified as a first closing portion 1136a and a second closing portion 1136b in FIG. 11A) and one or more closing levers or actuators 1138 (individually identified as a first closing lever 1138a and a second closing lever 1138b in FIG. 11A). Each of the closing portions 1136 can be positioned to control fluid flow through the lumen 204. Each of the closing levers 1138 can be operably coupled to at least one of the closing portions 1136, such that movement/rotation of the closing levers 1138 (as indicated by arrows F in FIG. 11A) can cause corresponding movement/rotation of the closing portions 1136, as indicated by arrows G in FIG. 11A. In the illustrated embodiment, for example, the first closing lever 1138a is operably coupled to the first closing portion 1136a and configured to cause movement/rotation thereof, and the second closing lever 1138b is operably coupled to the second closing portion 1136b and configured to cause movement/rotation thereof. One or more of the closing levers 1138 can be moved/rotated to change (e.g., increase or decrease) the flow of fluid through the lumen 204, for example, by positioning the closing portions 1136 to change (e.g., increase or decrease) a resistance to fluid flow through the lumen 204. In the illustrated embodiment, for example, rotation of the closing levers 1138 in a first direction (e.g., indicated by arrows F) can cause a corresponding rotation of the closing portions 1136 (e.g., indicated by arrows G) to increase a resistance to fluid flow through the lumen 204 and at least partially or fully prevent fluid flow therethrough. Rotation of the closing levers 1138 in a second direction (e.g., opposite the direction indicated by arrows F) can cause a corresponding rotation of the closing portions 1136 (e.g., opposite the direction indicated by arrows G) to decrease a resistance to fluid flow through the lumen 204 and increase fluid flow therethrough.

FIG. 11B is an illustration of the system 1100 of FIG. 11A. In some embodiments, the shunting element 202 further includes one or more tethers 1140 (individually identified as a first tether 1140a and a second tether 1140b in FIG. 11B). The tethers 1140 can include one or more polymers, fibers (e.g., fabric or woven fibers), sutures, and/or other suitable materials. Each of the tethers 1140 can be configured to at least partially or fully prevent movement/rotation of one of the closing levers 1138. In the illustrated embodiment, for example, the first tether 1140a is coupled to the first closing lever 1138a and configured to at least partially or fully prevent movement/rotation of the first closing lever 1138a in the direction indicated by arrow F, and the second tether 1140b is couple to the second closing lever 1138b and configured to at least partially or fully prevent movement/rotation of the second closing lever 1138b in the direction indicated by arrow F. As described previously with reference to FIG. 11A, the movement/rotation of the closing levers 1138 can cause corresponding movement/rotation of the closing portions 1136. Accordingly, at least partially or fully prevent movement/rotation of the closing levers 1138 can at least partially or fully prevent movement/rotation of the closing portions 1136, e.g., to at least partially or fully prevent changes to a resistance to fluid flow through the lumen 204.

The tethers 1140 can be configured to release the corresponding closing levers 1138 to allow movement/rotation thereof and/or of the closing portions 1136. In at least some embodiments, for example, or one or more of the tethers 1140 can be disconnected or severed to allow movement/rotation of the closing levers 1138 and/or the closing portions 1136. Additionally, or alternatively, one or more of the tethers 1140 can be configured to automatically release the corresponding closing lever 1138, such as after a predetermined amount of time has elapsed since implantation of the shunting element 202. For example, one or more of the tethers 1140 can be formed from a bioabsorbable material such that the tether 1140 decays after implantation of the shunting element 202.

In some embodiments, at least one of the tethers 1140 can be comprised of a shape memory material. In such embodiments, activation of a material phase change in one or more of the tethers 1140 (e.g., via heating) can induce a geometric change that releases and/or otherwise allows movement of the corresponding closing lever 1138. Following this, the closing lever 1138 can be returned toward and/or to its initial position (e.g., if a patient condition changes and shunt flow needs to be increased), such as by mechanically manipulating the closing lever 1138 and/or the corresponding tether 1140.

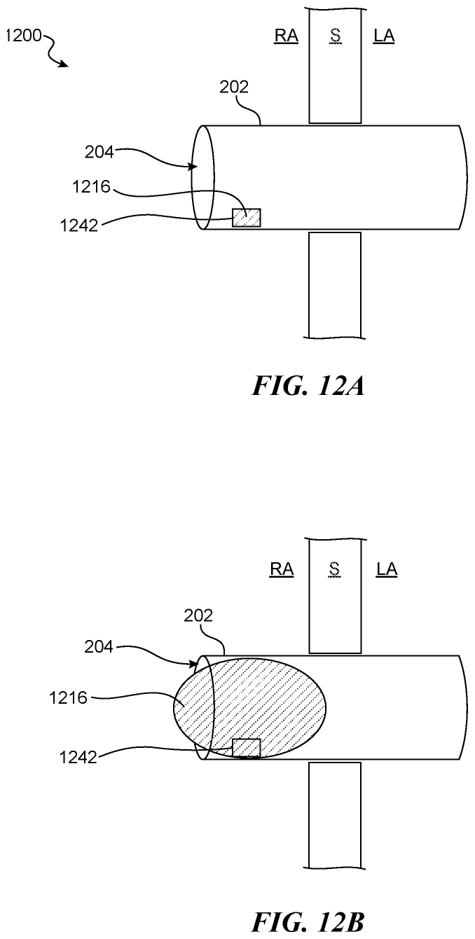

FIGS. 12A and 12B are illustrations of an interatrial shunting system 1200 (“system 1200”) configured in accordance with embodiments of the present technology. The system 1200 can include at least some features that are at least generally similar or identical in structure and/or function to the system 200 of FIG. 2. In at least some embodiments, for example, the system 1200 includes the shunting element 202 defining the lumen 204 therethrough. Additionally, the system 1200 further includes a container or cartridge 1242 configured to contain a sealing component or compound 1216. The sealing compound 1216 can include an expandable and/or inflatable foam (e.g., open/closed cell foams, flexible/memory foams, etc.). These foams can be comprised in part or in whole of ethylene copolymer, expanded polyethylene, polyethylene, polyurethane, polystyrene, polycarbonate, polyester, polyether, polyetherimide, polyimide, and/or another suitable material. In some embodiments, the sealing compound 1216 can include an expandable wire mesh, coil, and/or similar structure that can be movably coupled to a membrane, foam, and/or other material. The cartridge 1242 can be positioned at least partially within the lumen 204 and configured to release the sealing compound 1216 to allow the sealing compound 1216 to expand to at least partially or fully fill a cross-sectional area of the lumen 204. The expansion of the sealing compound 1216 can at least partially or fully prevent fluid flow through the lumen 204. In some embodiments, the cartridge 1242 can be configured to release the sealing compound 1216 in response to energy, such as received from the energy transmission device(s) 208 in FIG. 2. Additionally, or alternatively, the cartridge 1242 can be configured to release the sealing compound 1216 automatically, such as after a predetermined amount of time (e.g., after implantation of the shunting element 202) has elapsed. In these and other, the cartridge 1242 can be configured to release the sealing compound 1216 in response to a mechanical stimulus delivered by an operator, or after a deliberate action of a healthcare provider.

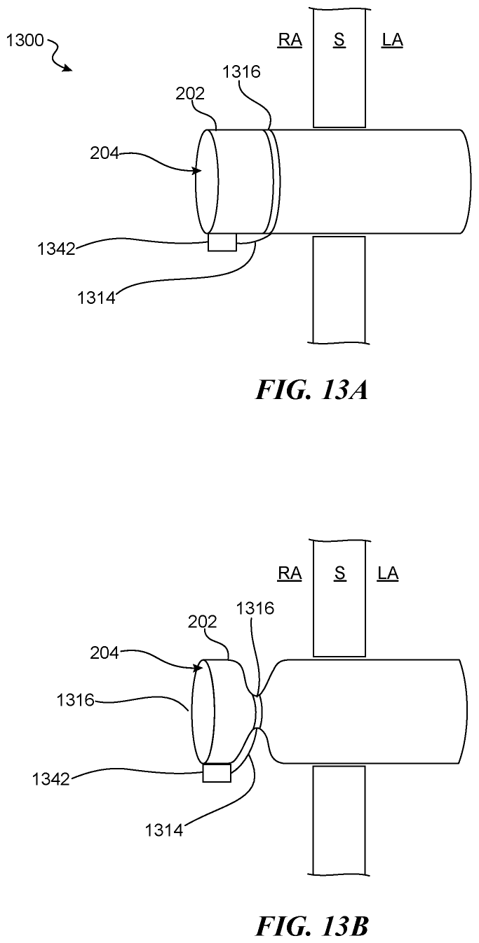

FIGS. 13A and 13B are illustrations of an interatrial shunting system 1300 (“system 1300”) configured in accordance with embodiments of the present technology. The system 1300 can include at least some features that are at least generally similar or identical in structure and/or function to the system 200 of FIG. 2. In at least some embodiments, for example, the system 1300 includes the shunting element 202 defining the lumen 204 therethrough. The system 1300 further includes a sealing component 1316 positioned around at least a portion of the shunting element 202. The sealing component 1316 can be at least generally similar or identical in structure and/or function to the sealing component 816 of FIGS. 8A and 8B. Additionally, the system 1300 can include a cartridge 1342 coupled to the shunting element 202 (e.g., an exterior of the shunting element 202), coupled to and/or embedded at least partially within the septal wall S, positioned in the RA, or at another suitable location with the patient. The cartridge 1342 can be operably coupled to the sealing component 1316 by one or more tethers 1314. The cartridge 1342 can be activated (e.g., mechanically, via heat energy, etc.) to cause the sealing component 1316 to cinch or tighten around the shunting element 202 to reduce a diameter/shape of the lumen 204 and/or otherwise at least partially or fully prevent fluid flow therethrough. In the illustrated embodiment, for example, the cartridge 1342 is configured to retract the one or more tethers 1314 to cause the sealing component 1316 to cinch/tighten around the shunting element 202.

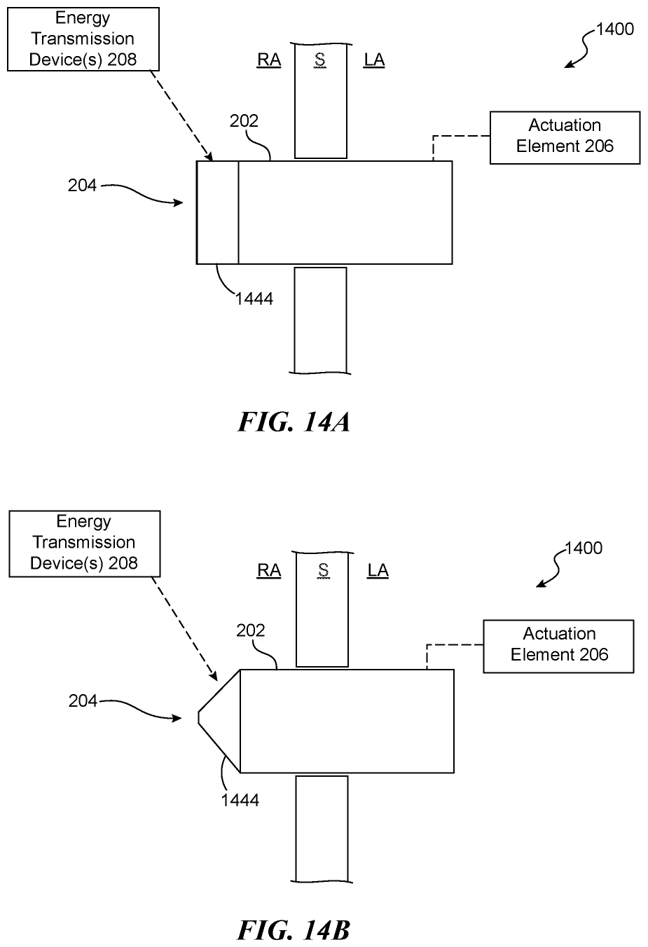

FIGS. 14A and 14B are partially-schematic illustrations of an interatrial shunting system 1400 (“system 1400”) configured in accordance with embodiments of the present technology. The system 1400 can include at least some features that are at least generally similar or identical in structure and/or function to the system 200 of FIG. 2. In at least some embodiments, for example, the system 1400 includes the shunting element 202 defining the lumen 204. Additionally, the shunting element 202 can include a closing or restricting portion 1444 configured to be transitionable between (i) a first state or configuration in which the restricting portion 1444 provides a first resistance to fluid flow through the lumen 204 and/or otherwise does not substantially prevent fluid flow through the lumen 204 (e.g., FIG. 13A), and (ii) a second state or configuration in which the restricting portion 1444 provides a second resistance to fluid flow through the lumen 204 higher than the first resistant and/or otherwise at least partially or fully prevents fluid flow through the lumen 204 (e.g., FIG. 13B). The restricting portion 1444 can be configured to transition between the first and second states in response to energy (e.g., received from the energy transmission device(s) 208 in FIG. 2), which can be provided by one or more of the energy transmission devices 208. In at least some embodiments, for example, the restricting portion 1444 can be formed from a shape memory material or alloy, such as nitinol or another suitable material. In some embodiments, energy supplied to transition the restricting portion 1444 between the first and second states can be first energy, and energy supplied to the actuation element 206 can be second energy having one or more parameters (e.g., amplitude, frequency, power/pulse energy, etc.) different than the first energy. In at least some embodiments, for example, the restricting portion 1444 can be configured to transition between the first and second states in response to first energy having a greater pulse energy than the second energy at which the actuation element 206 is configured to transition, such that actuation of the actuation element 206 with the second energy is not expected to result in actuation of the restricting portion 1444. The first energy can be applied directly to the restricting portion 1444, as shown in FIGS. 14A and 14B, or applied to another portion of the shunting element 202 and transmitted to the restricting portion 1444. As one of skill in the art will appreciate from the disclosure herein, various components of the interatrial shunting systems described above can be omitted without deviating from the scope of the present technology. Likewise, additional components not explicitly described above may be added to the interatrial shunting systems without deviating from the scope of the present technology. Accordingly, the present technology is not limited to the configurations expressly identified herein, but rather encompasses variations and alterations of the described systems.

### EXAMPLES

Several aspects of the present technology are described with reference to the following examples:
1. An implantable shunting system, comprising:
   an interatrial shunt configured to be positioned across a septal wall of a heart of a patient, wherein the interatrial shunt defines a lumen extending therethrough; and
   a plug having a first end portion and a second end portion opposite the first end portion,
   wherein the plug is configured to be positioned at least partially within the lumen in vivo with the first end portion on a first side of the septal wall and/or the second end portion on a second side of the septal wall opposite the first side, and
   wherein the plug is configured to at least partially prevent fluid flow through the lumen when positioned at least partially within the lumen.
2. The implantable shunting system of example 1 wherein the first side is within a right atrium of the heart of the patient and wherein the second side is within a left atrium of the heart of the patient.
3. The implantable shunting system of example 1 or 2 wherein the plug includes an expandable body configured to expand within the lumen to at least partially movement the plug from moving relative to the interatrial shunt.
4. The implantable shunting system of any of examples 1-4 wherein the plug includes one or more retention components configured to operably engage at least a portion of the septal wall to at least partially prevent the plug from moving relative to the interatrial shunt.
5. The implantable shunting system of any of examples 1-4 wherein the plug includes one or more retention components configured to operably engage at least a portion of the interatrial shunt to at least partially prevent the plug from moving relative to the interatrial shunt.
6. The implantable shunting system of example 5 wherein the one or more retention components include one or more arms, one or more loops, and/or one or more springs.
7. The implantable shunting system of example 5 or 6 wherein the plug includes a body, and wherein the one or more retention components extend radially outwardly from the body.
8. The implantable shunting system of example 7 wherein the interatrial shunt includes an inner portion that at least partially defines the lumen, and wherein the one or more retention components are configured to contact the inner portion of the interatrial shunt.
9. The implantable shunting system of any of examples 1-8 wherein the interatrial shunt includes a left atrial portion configured to be positioned in a left atrium of the heart, and wherein the second end portion of the plug includes one or more arms configured to contact the left atrial portion of the interatrial shunt to at least partially prevent the plug from moving relative to the interatrial shunt.
10. The implantable shunting system of any of examples 1-9 wherein the interatrial shunt includes a right atrial portion configured to be positioned in a right atrium of the heart, and wherein the first end portion of the plug includes one or more arms configured to contact the right atrial portion of the interatrial shunt to at least partially prevent the plug from moving relative to the interatrial shunt.
11. The implantable shunting system of any of examples 1-10 wherein the first end portion of the plug includes one or more arms configured to contact a left atrial side of the septal wall to at least partially prevent the plug from moving relative to the interatrial shunt.
12. The implantable shunting system of any of examples 1-11 wherein the plug includes one or more temperature sensors, flow sensors, conductivity sensors, and/or pressure sensors.
13. The implantable shunting system of any of examples 1-12 wherein the first end portion of the plug includes a first sensor and the second end portion of the plug includes a second sensor.
14. An adjustable shunting system, comprising:
   an interatrial shunt configured to be positioned across a septal wall of a heart of a patient, wherein the interatrial shunt defines a perimeter and a lumen extending between a right atrium of the heart and a left atrium of the heart of the patient; and
   a closing device positioned to operably engage at least a portion of the perimeter, wherein the closing device is configured to apply a radially compressive force to the portion of the perimeter and thereby at least partially prevent fluid flow through the lumen.
15. The adjustable shunting system of example 14 wherein the closing device includes:
   a loop configured to extend around at least the portion of the perimeter and cinch closed around the portion of the perimeter to apply the radially compressive force;
   an anchor configured to be positioned at least partially within the septal wall; and
   a tether coupling the loop to the anchor.
16. The adjustable shunting system of example 14 or 15 wherein the interatrial shunt includes a closure retention feature extending radially outwardly from the lumen to define a closure capture region configured to receive the closing device and thereby at least partially prevent movement of the closing device relative to the interatrial shunt.
17. The adjustable shunting system of any of examples 14-16 wherein the closing device includes a cap defining an opening and one or more retention teeth positioned about the opening, and wherein:
   the cap is configured to receive at least the portion of the perimeter through the opening; and
   the one or more retention teeth are configured to operably engage the interatrial shunt to at least partially prevent movement of the closing device relative to the interatrial shunt.
18. An adjustable shunting system, comprising:
   an interatrial shunt configured to be positioned across a septal wall of a heart of a patient, the interatrial shunt having a first end portion, a second end portion opposite the first end portion, and defining a lumen extending therebetween;
   an actuation element operably coupled to the interatrial shunt and configured to adjust a diameter of the lumen through a first range of adjustment to adjust fluid flow therethrough; and
   one or more stopping components operably coupled to the interatrial shunt to reduce the adjustability of the diameter of the lumen to a second range of adjustment, different than the first range of adjustment.
19. The adjustable shunting system of example 18 wherein the second range of adjustment is less than the first range of adjustment such that, independent of a state of the actuation element, fluid is expected to flow through the lumen when the one or more stopping components reduce the adjustability to the second range of adjustment.
20. The adjustable shunting system of example 18 or 19 wherein the one or more stopping components include a shape memory material element extending circumferentially about the lumen, wherein the one or more stopping components are configured to transition between (i) a first configuration in which the shape memory material element reduces the adjustability of the diameter to the second range of adjustment, and (ii) a second configuration in which the shape memory material element returns the adjustability of the diameter to the first range of adjustment.

### Conclusion

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable shunting system, comprising:
an interatrial shunt configured to be positioned across a septal wall of a heart of a patient, wherein the interatrial shunt defines a lumen extending therethrough; and
a plug having a first end portion and a second end portion opposite the first end portion,
wherein the plug is configured to be positioned at least partially within the lumen in vivo with the first end portion on a first side of the septal wall and/or the second end portion on a second side of the septal wall opposite the first side, and
wherein the plug is configured to at least partially prevent fluid flow through the lumen when positioned at least partially within the lumen.

2. The implantable shunting system of claim 1 wherein the first side is within a right atrium of the heart of the patient and wherein the second side is within a left atrium of the heart of the patient.

3. The implantable shunting system of claim 1 wherein the plug includes an expandable body configured to expand within the lumen to at least partially movement the plug from moving relative to the interatrial shunt.

4. The implantable shunting system of claim 1 wherein the plug includes one or more retention components configured to operably engage at least a portion of the septal wall to at least partially prevent the plug from moving relative to the interatrial shunt.

5. The implantable shunting system of claim 1 wherein the plug includes one or more retention components configured to operably engage at least a portion of the interatrial shunt to at least partially prevent the plug from moving relative to the interatrial shunt.

6. The implantable shunting system of claim 5 wherein the one or more retention components include one or more arms, one or more loops, and/or one or more springs.

7. The implantable shunting system of claim 5 wherein the plug includes a body, and wherein the one or more retention components extend radially outwardly from the body.

8. The implantable shunting system of claim 7 wherein the interatrial shunt includes an inner portion that at least partially defines the lumen, and wherein the one or more retention components are configured to contact the inner portion of the interatrial shunt.

9. The implantable shunting system of claim 1 wherein the interatrial shunt includes a left atrial portion configured to be positioned in a left atrium of the heart, and wherein the second end portion of the plug includes one or more arms configured to contact the left atrial portion of the interatrial shunt to at least partially prevent the plug from moving relative to the interatrial shunt.

10. The implantable shunting system of claim 1 wherein the interatrial shunt includes a right atrial portion configured to be positioned in a right atrium of the heart, and wherein the first end portion of the plug includes one or more arms configured to contact the right atrial portion of the interatrial shunt to at least partially prevent the plug from moving relative to the interatrial shunt.

11. The implantable shunting system of claim 1 wherein the first end portion of the plug includes one or more arms configured to contact a left atrial side of the septal wall to at least partially prevent the plug from moving relative to the interatrial shunt.

12. The implantable shunting system of claim 1 wherein the plug includes one or more temperature sensors, flow sensors, conductivity sensors, and/or pressure sensors.

13. The implantable shunting system of claim 1 wherein the first end portion of the plug includes a first sensor and the second end portion of the plug includes a second sensor.

14. An adjustable shunting system, comprising:
an interatrial shunt configured to be positioned across a septal wall of a heart of a patient, wherein the interatrial shunt defines a perimeter and a lumen extending between a right atrium of the heart and a left atrium of the heart of the patient; and
a closing device positioned to operably engage at least a portion of the perimeter, wherein the closing device is configured to apply a radially compressive force to the portion of the perimeter and thereby at least partially prevent fluid flow through the lumen.

15. The adjustable shunting system of claim 14 wherein the closing device includes:
a loop configured to extend around at least the portion of the perimeter and cinch closed around the portion of the perimeter to apply the radially compressive force;
an anchor configured to be positioned at least partially within the septal wall; and
a tether coupling the loop to the anchor.

16. The adjustable shunting system of claim 14 wherein the interatrial shunt includes a closure retention feature extending radially outwardly from the lumen to define a closure capture region configured to receive the closing device and thereby at least partially prevent movement of the closing device relative to the interatrial shunt.

17. The adjustable shunting system of claim 14 wherein the closing device includes a cap defining an opening and one or more retention teeth positioned about the opening, and wherein:
the cap is configured to receive at least the portion of the perimeter through the opening; and
the one or more retention teeth are configured to operably engage the interatrial shunt to at least partially prevent movement of the closing device relative to the interatrial shunt.

18. An adjustable shunting system, comprising:
an interatrial shunt configured to be positioned across a septal wall of a heart of a patient, the interatrial shunt having a first end portion, a second end portion opposite the first end portion, and defining a lumen extending therebetween;
an actuation element operably coupled to the interatrial shunt and configured to adjust a diameter of the lumen through a first range of adjustment to adjust fluid flow therethrough; and
one or more stopping components operably coupled to the interatrial shunt to reduce the adjustability of the diameter of the lumen to a second range of adjustment, different than the first range of adjustment.

19. The adjustable shunting system of claim 18 wherein the second range of adjustment is less than the first range of adjustment such that, independent of a state of the actuation element, fluid is expected to flow through the lumen when the one or more stopping components reduce the adjustability to the second range of adjustment.

20. The adjustable shunting system of claim 18 wherein the one or more stopping components include a shape memory material element extending circumferentially about the lumen, wherein the one or more stopping components are configured to transition between (i) a first configuration in which the shape memory material element reduces the adjustability of the diameter to the second range of adjustment, and (ii) a second configuration in which the shape memory material element returns the adjustability of the diameter to the first range of adjustment.
